(19) United States
(12) Patent Application Publication
(10) Pub. No.: US 2005/0048048 A1
(43) Pub. Date: Mar. 3, 2005

(54) Odor absorbing pad for shoes

(76) Inventor: Jaime Valenzuela, Los Angeles, CA (US)

Correspondence Address: Cary Tope McKay, Suite 311, 23852 Pacific Coast Highway, Malibu, CA, 90265, US

(21) Appl. No.: 10/653,686

(22) Filed: Sep. 2, 2003

(51) Int. Cl. A61K 33/44; B67D 5/42; G01F 11/00

(52) U.S. Cl. 424/125; 222/386

(57) ABSTRACT

The present invention relates to odor absorbing pads for shoes. More particularly, it relates to odor absorbing pads having a spring-loaded powder dispersion system. The spring-loaded powder dispersion system has a springing means, whereby when actuated, the springing means causes a powder delivery medium to release a specific powder which is passed through a breathable aperture into a foot compartment of a shoe where it is distributed about a foot.

### FIELD OF INVENTION

The present invention relates to odor absorbing pads for shoes and more particularly, to odor absorbing pads for removing odor from shoes.

### BACKGROUND OF INVENTION

The use of shoe liners is known in prior art. More specifically, shoe liners heretofore devised and utilized for a purpose of providing comfort to a user and removing odor are known to consist basically of familiar, expected and obvious structural configurations. By way of example, the prior art includes U.S. Pat. No. 5,399,404; U.S. Pat. No. 4,864,740; U.S. Pat. No. 4,257,176; U.S. Pat. No. 4,185,402; and U.S. Pat. No. 5,261,169.

Odor absorbing pads often contain a desired specific powder. The specific powder may be an anti-fungal powder, an anti-odor powder, a medicinal powder, or a scented powder. Although the specific powder will serve its purpose if released from the odor-absorbing pad, it is often trapped therein. The odor absorbing pads previously devised have no means of actuating the specific powder and thereby forcing it into a foot compartment of a shoe.

Therefore, it can be appreciated that there exists a continuing need for a new and improved odor absorbing pads for shoes with a springing means to actuate and release a specific powder. In this regard, the present invention substantially fulfills this need.

### SUMMARY OF INVENTION

The odor absorbing pad in the present invention comprises a spring-loaded powder dispersion system for dispersing powder into a foot compartment of a shoe. The spring-loaded powder dispersion system comprises a top portion with a breathable aperture therein, an air passage portion, an elastically deformable housing, and a springing means.

The air passage portion has elastically deformable sidewalls, an open top part, and an open bottom part. The open top part and the open bottom part are substantially separated from one another and the open top part is in fluid communication with the breathable aperture of the top portion. Additionally, the air passage portion selectively allows for air to be stored in the air passage portion or for air to pass through the air passage portion.

The elastically deformable housing has a top portion and a bottom portion. The top portion of the elastically deformable housing is in air-permeable communication with the open bottom part of the air passage portion. Furthermore, the elastically deformable housing contains a powder delivery medium.

The springing means is in contact with the bottom portion of the elastically deformable housing. When the spring-loaded powder dispersion system is actuated, the springing means moves the elastically deformable housing, causing the powder delivery medium to release a specific powder. The specific powder is then passed from the elastically deformable housing through the breathable aperture into a foot compartment of a shoe, where it is distributed about a foot.

In another aspect, the powder delivery medium is selected from a group consisting of charcoal and baking soda.

In yet another aspect, the specific powder includes a powder selected from a group consisting of an anti-fungal powder, an anti-odor powder, a medicinal powder, and a scented powder.

Additionally, the top portion is substantially circular in shape and the breathable aperture is made of a porous material containing micro-holes.

Furthermore, the elastically deformable housing is constructed of plastic and comprises a plurality of compartments. Any compartment is interchangeable with any other compartment in the plurality of compartments. Additionally, each compartment contains a powder delivery medium with a specific powder, where the specific powder of a compartment is different from the specific powder of other compartments. Moreover, a compartment with a specific powder content may be placed in the elastically deformable housing at any position.

In yet another aspect, the elastically deformable housing is configured such that a compartment containing a specific powder can be manually removed from the elastically deformable housing and replaced with another compartment containing a different specific powder.

In addition, the spring-loaded dispersion system is formed such that it is detachably attachable with a sole of a shoe or such that it is permanently attachable with a sole of a shoe.

In another aspect, when the spring-loaded powder dispersion system is actuated, the springing means, the elastically deformable housing, the air passage portion, and the top portion are configured to generate an airflow through the spring-loaded powder dispersion system, thereby significantly preventing moisture from penetrating into the elastically deformable housing.

In yet another aspect, a height of the top portion is less than about 1/16 of an inch, a height of the air passage portion is less than about ⅛ of an inch, a height of the elastically deformable housing is less than about ⅛ of an inch, and a height of the springing means is less than about ¼ of an inch.

Furthermore, a moisture absorbent portion is attached above the top portion. The moisture absorbent portion reduces an amount of moisture penetrating into the spring-loaded powder dispersion system. The moisture absorbent portion has a center aperture, allowing free passage of air and powder from the spring-loaded powder dispersion system into the foot compartment of a shoe. Additionally, the moisture absorbent portion is filled with a highly absorbent material surrounded by a hydrophobic material in order to absorb and retain a substantial part of any moisture generated by the foot.

In another aspect, the spring-loaded powder dispersion system comprises a pair of spring-loaded powder dispersion systems. The pair has a front system and a rear system, with the front system having a first springing means with a first bottom face and the rear system having a second springing means with a second bottom face. The first bottom face and the second bottom face have a layer of adhesive formed thereon. Through the layer of adhesive, the first springing means is adhered with a top surface of a sole of a shoe near a front end thereof and a second springing means is adhered with the top surface of the sole of the shoe near a rear end thereof.

In yet another aspect, the springing means is coated with an anticorrosive material.

In another aspect, the odor absorbing pad comprises a powder dispersion system for dispersing powder into a foot compartment of a shoe, where the shoe contains a springing means imbedded into an interior of a sole of the shoe. The powder dispersion system comprises a top portion having a breathable aperture therein, an air passage portion, an elastically deformable housing, and a springing means embedded into the interior of a sole of a shoe

In this aspect, the springing means is embedded into the interior of a sole of a shoe. The springing means is in fluid communication with the bottom portion of the elastically deformable housing. When the powder dispersion system is actuated, the springing means moves the elastically deformable housing, causing the powder delivery medium to release a specific powder. The specific powder is then passed from the elastically deformable housing and through the breathable aperture into a foot compartment of a shoe where it is distributed about a foot.

In another aspect, the spring-loaded dispersion system comprises a top portion having a breathable aperture therein, an air passage portion, and an elastically deformable housing. The elastically deformable housing has a top portion and a bottom portion. The top portion of the housing is in air-permeable communication with the open bottom part of the air passage portion. Additionally, the housing contains a power delivery medium and a springing means. When the spring-loaded powder dispersion system is actuated, the springing means pushes air through the elastically deformable housing, causing the powder delivery medium to release a specific powder. The specific powder is then passed from the elastically deformable housing through the breathable aperture into a foot compartment of a shoe where it is distributed about a foot.

In another aspect, the springing means imbedded into the elastically deformable housing is coated with an anticorrosive material.

In another aspect, the present invention comprises a method for forming a spring-loaded powder dispersion system. The method comprises acts of selecting a top portion with a breathable aperture; selecting an air passage portion having elastically deformable side walls, an open top part, and an open bottom part, where the open top part and the open bottom part are substantially separated from one another; attaching the open top part of the air passage portion with the breathable aperture of the top portion; selecting an elastically deformable housing having a top portion, a bottom portion, and containing a powder delivery medium; attaching the top portion of the elastically deformable housing with the open bottom part of the air passage portion; selecting a springing means; and attaching the spring means with the bottom portion of the elastically deformable housing.

In another aspect, the method for forming a spring-loaded powder dispersion system further comprises acts of selecting a moisture absorbent portion and attaching the moisture absorbent portion with the top portion. The moisture absorbent portion reduces an amount of moisture penetrating into the spring-loaded powder dispersion system.

In yet another aspect, the method for forming a spring-loaded powder dispersion further comprises an act of adding an additional spring-loaded powder dispersion system, thereby comprising a pair of spring-loaded powder dispersion systems. The pair has a front system and a rear system. Additionally, the front system has a first springing means with a first bottom face and the rear system has a second springing means with a second bottom face.

In another aspect, the first springing means and the elastically deformable housing have a springing diameter and a housing diameter, and the springing diameter may be greater than the housing diameter.

In another aspect, the front system and the rear system have a front height and a rear height, and the rear height may be greater than the front height.

Finally, in the act of adding an additional spring-loaded powder dispersion system, the first bottom face and the second bottom face have a layer of adhesive formed thereon. With the layer of adhesive, the first springing means is attached with a top surface of a sole of a shoe near a front end thereof and a second springing means is attached with the top surface of the sole of the shoe near a rear end thereof.

### BRIEF DESCRIPTION OF THE DRAWINGS

The nature of the odor absorbing pads described herein will be readily apparent in the following drawings, in which:

FIG. 1A is a cross-sectional view of the present invention, illustrating each section of a spring-loaded powder dispersion system;

FIG. 1B is a top perspective view of the present invention, illustrating a top portion having a breathable aperture therein;

FIG. 2 is a side perspective of the present invention, illustrating the inter-relation between each section;

FIG. 3A is a top perspective view of the present invention, illustrating a moisture absorbent portion with a pair of spring-loaded powder dispersion systems attached thereto;

FIG. 3B is a side perspective view of the present invention, illustrating the moisture absorbent portion with the pair of spring-loaded powder dispersion systems attached thereto;

FIG. 4 is a top view of the present invention, showing the pair of spring-loaded powder dispersion systems positioned within a shoe;

FIG. 5 is a side perspective view of the present invention, showing the pair of spring-loaded powder dispersion systems positioned within the shoe;

FIG. 6 is a top perspective view of the present invention, illustrating another aspect, where the shoe contains a springing means imbedded into a sole of the shoe; and

FIG. 7 is side perspective view of the present invention, illustrating another aspect, where the shoe contains a springing means imbedded into the sole of the shoe.

### DETAILED DESCRIPTION

The present invention relates to odor absorbing pads for shoes. More particularly, it relates to odor absorbing pads having a spring-loaded powder dispersion system. The spring-loaded powder dispersion system has a springing means, whereby when actuated, the springing means causes a powder delivery medium to release a specific powder which is passed through a breathable aperture into a foot compartment of a shoe where it is distributed about a foot.

The following description, taken in conjunction with the referenced drawings, is presented to enable one of ordinary skill in the art to make and use the invention. Various modifications will be readily apparent to those skilled in the art, and the general principles defined herein may be applied to a wide range of aspects. Thus, the present invention is not intended to be limited to the aspects presented, but is to be accorded the widest scope consistent with the principles and novel features disclosed herein. Furthermore it should be noted that unless explicitly stated otherwise, the figures included herein are illustrated diagrammatically and without any specific scale, as they are provided as qualitative illustrations of the concept of the present invention.

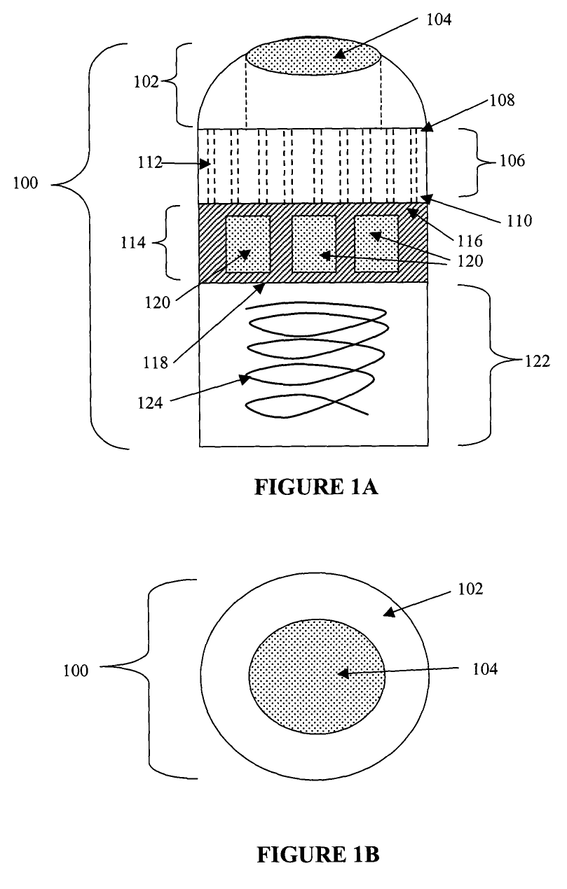

Referring to the figures, FIG. 1A illustrates an aspect of the spring-loaded powder dispersion system 100 in accordance with the present invention. The spring-loaded powder dispersion system 100 has a top portion 102 with a breathable aperture 104 therein. The top portion 102 may be any suitable shape. For example, from a top perspective view, the top portion 102 may be substantially circular in shape, or from a side perspective view, may have a dome. Additionally, the top portion may be constructed of any suitable material, a non-limiting example of which includes polyurethane. The breathable aperture 104 may be constructed of any suitably porous material containing micro-holes.

The spring-loaded powder dispersion system 100 also has an air passage portion 106. The air passage portion 106 has elastically deformable sidewalls, an open top part 108, an open bottom part 110, and micro-holes 112. The air passage portion 106 may be constructed of any suitable material, a non-limiting example of which includes plastic. The open top part 108 and the open bottom part 110 are substantially separated from one another. Additionally, the open top part 108 is in fluid communication with the breathable aperture 104 of the top portion 102, such that the air passage portion 106 selectively allows for air to be stored in the air passage portion 106 or for air to pass through the air passage portion 106 through the micro-holes 112.

In communication with the air passage portion 106 is an elastically deformable housing 114. The elastically deformable housing 114 has a top portion 116 and a bottom portion 118. The top portion 116 is in air-permeable communication with the open bottom part 110 of the air passage portion 106. The elastically deformable housing 114 may be constructed of any suitable material, non-limiting examples of which include plastic or polyurethane.

Additionally, the elastically deformable housing 114 contains a plurality of manually interchangeable compartments 120. Each compartment 120 contains a powder delivery medium with a specific powder. The powder delivery medium may be any suitable medium for delivering a powder, non-limiting examples of which include charcoal and baking soda. The specific powder may be any powder providing a desirable result, non-limiting examples of which include anti-fungal powder, an anti-odor powder, a medicinal powder, and a scented powder.

In contact with the bottom portion 118 of the elastically deformable housing 114 is a springing means 122. The springing means 122 may be any suitable means for creating an expansive force, a non-limiting example of which includes a metal spring 124. The springing means 122 may be coated with an anti corrosive material. When the spring-loaded powder dispersion system 100 is actuated, the springing means 122 moves the elastically deformable housing 114, causing the powder delivery medium to release a specific powder.

Each section may be any suitable size or height. For example, a height of the top portion 102 may be less than about 1/16 of an inch, a height of the air passage portion 106 may be less than about ⅛ of an inch, a height of the elastically deformable housing 114 may be less than about ⅛ of an inch, and a height of the springing means 122 may be less than about ¼ of an inch.

Illustrated in FIG. 1B is a top-perspective view of the spring-loaded powder dispersion system 100. As shown in this perspective, the spring-loaded powder dispersion system 100 may be substantially circular in shape. Additionally, the breathable aperture 104 of the top portion 102 is accessible from the top-most portion of the spring-loaded powder dispersion system 100.

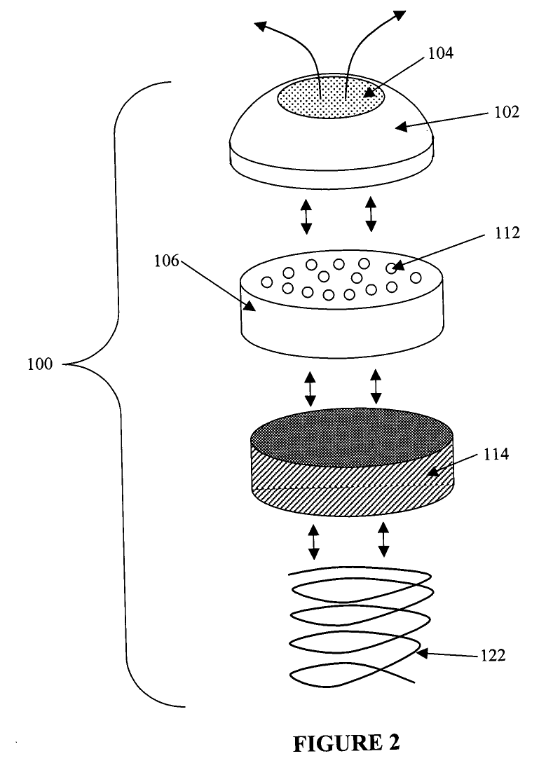

FIG. 2 illustrates the interrelation between each section. As shown in FIG. 2, the springing means 122, the elastically deformable housing 114, the air passage portion 116, and the top portion 102 are configured to generate airflow through the spring-loaded powder dispersion system 100, thereby preventing moisture from penetrating into the elastically deformable housing 114. When the spring-loaded powder dispersion system 100 is actuated, the springing means 122 moves the elastically deformable housing 114, causing the powder delivery medium to release a specific powder. The specific powder is then passed from the elastically deformable housing 114 and through the micro-holes 112 in the air passage portion 106. The specific powder then passes through the breathable aperture 104 into a foot compartment of a shoe where it is distributed about a foot.

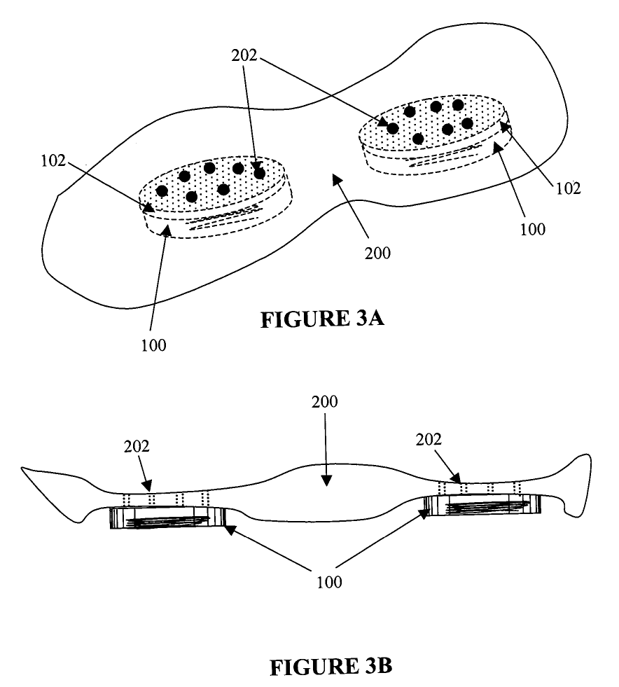

Illustrated in FIG. 3A is a pair of spring-loaded powder dispersion systems 100. As shown in this aspect, a moisture absorbent portion 200 is attached above the top portion 102 of each spring-loaded powder dispersion system 100, thereby reducing an amount of moisture from penetrating into the spring-loaded powder dispersion system 100. The moisture absorbent portion 200 has a center aperture 202 allowing free passage of air and specific powder from the spring-loaded dispersion system 100 into the foot compartment of a shoe. The moisture absorbent portion 200 may be filled with a highly absorbent material surrounded by a hydrophobic material, effectively absorbing and retaining a substantial part of any moisture generated by a foot.

Illustrated in FIG. 3B is a side perspective view of the moisture absorbent portion 200 attached with a pair of spring-loaded powder dispersion systems 100. As shown, the spring-loaded powder dispersion systems 100 are in fluid communication with the center apertures 202, allowing the specific powder to pass from the spring-loaded powder dispersion systems 100, through the center apertures 202 and into the foot compartment of the shoe.

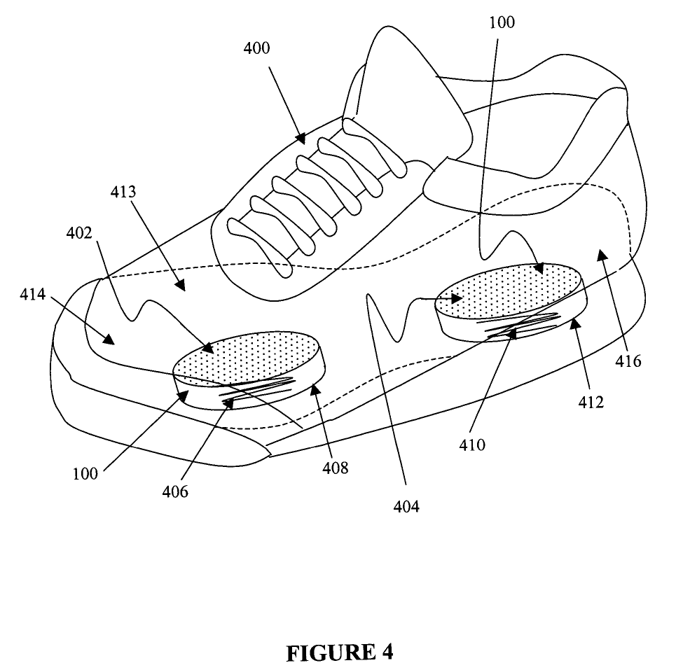

Illustrated in FIG. 4 is a pair of spring-loaded powder dispersion systems 100 placed within a shoe 400. The spring-loaded powder dispersion systems 100 are formed such that they may be detachably attachable with a sole of the shoe, or may be permanently attached. When permanently attached, the pair has a front system 402 and a rear system 404. The front system 402 has a first springing means 406 with a first bottom face 408 and the rear system 404 has a second springing means 410 with a second bottom face 412. The first bottom face 408 and the second bottom face 412 have a layer of adhesive formed thereon. The first springing means 406 is adhered with a top surface of a sole 413 of the shoe 400 near a front end 414 thereof. Furthermore, the second springing means 410 is adhered with a top surface of the sole 413 of the shoe 400 near a rear end 416 thereof.

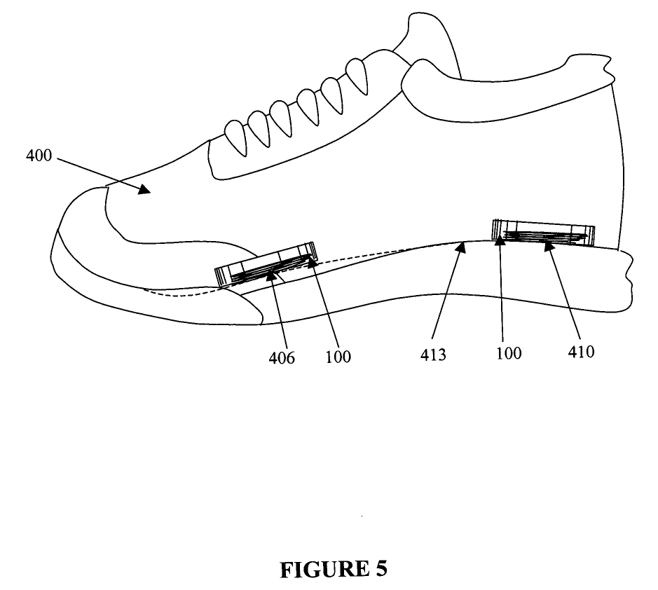

Illustrated in FIG. 5 is a side perspective view, showing the spring-loaded powder dispersion systems 100 attached with the top surface of a sole 413 of the shoe 400. As shown, the first springing means 406 and second springing means 410 are attached with a top surface of the sole 413 of the shoe 400. They may be either detachably attachable or permanently attached.

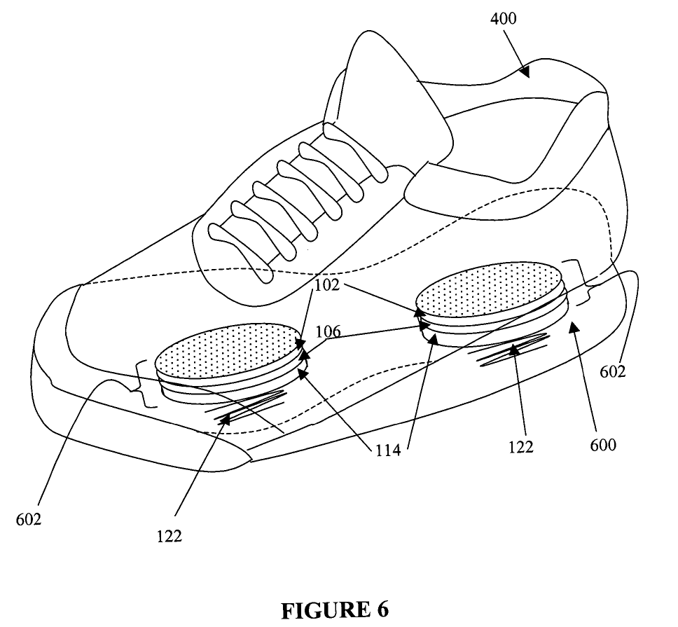

Illustrated in FIG. 6 is another aspect of the present invention. In this aspect, the shoe 400 contains a springing means 122 embedded into an interior of a sole 600 of the shoe 400. In fluid communication with the springing means 122 is a powder dispersion system 602. The powder dispersion system 602 has a top portion 102, an air passage portion 106, and an elastically deformable housing 114. When the powder dispersion system 602 is actuated, the springing means 122 moves the elastically deformable housing 114, causing a powder delivery medium to release a specific powder. The specific powder is then passed from the elastically deformable housing 114, through the powder dispersion system 602, and into a foot compartment of the shoe 400 where it is distributed about a foot.

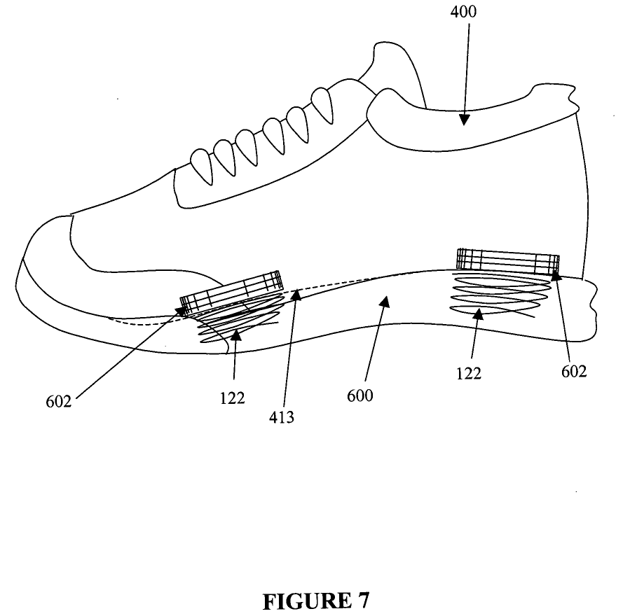

Illustrated in FIG. 7 is a side perspective view of another aspect of the present invention, showing a springing means 122 embedded into the interior of a sole 600 of the shoe 400. As shown, the powder dispersion systems 602 are in fluid communication with the top surface of the sole 413 of the shoe 400. They may be either detachably attachable or permanently attached.

1. A spring-loaded powder dispersion system for dispersing powder into a foot compartment of a shoe, the spring-loaded powder dispersion system comprising:
a top portion having a breathable aperture therein;
an air passage portion, the air passage portion having elastically deformable side walls, an open top part, and an open bottom part, where the open top part and the open bottom part are substantially separated from one another and the open top part is in fluid communication with the breathable aperture of the top portion, and wherein the air passage portion selectively allows for air to be stored in the air passage portion or for air to pass through the air passage portion;
an elastically deformable housing having a top portion and a bottom portion, with the top portion of the elastically deformable housing in air-permeable communication with the open bottom part of the air passage portion, the elastically deformable housing containing a powder delivery medium; and
a springing means in contact with the bottom portion of the elastically deformable housing, whereby when the spring-loaded powder dispersion system is actuated, the springing means moves the elastically deformable housing, causing the powder delivery medium to release a specific powder which is passed from the elastically deformable housing through the breathable aperture into a foot compartment of a shoe where it is distributed about a foot.

2. A spring-loaded powder dispersion system as set forth in claim 1, wherein the powder delivery medium is selected from a group consisting of charcoal and baking soda.

3. A spring-loaded powder dispersion system as set forth in claim 1, wherein the specific powder includes a powder selected from a group consisting of an anti-fungal powder, an anti-odor powder, a medicinal powder, and a scented powder.

4. A spring-loaded powder dispersion system as set forth in claim 1, wherein the top portion is substantially circular in shape and the breathable aperture is made of a porous material containing micro-holes.

5. A spring-loaded powder dispersion system as set forth in claim 1, wherein the elastically deformable housing is constructed of plastic and comprises a plurality of compartments.

6. A spring-loaded powder dispersion system as set forth in claim 5, wherein any compartment is interchangeable with any other compartment in the plurality of compartments, and where each compartment contains a powder delivery medium with a specific powder, where the specific powder of a compartment is different from the specific powder of other compartments, and where a compartment with a specific powder content may be placed in the elastically deformable housing at any position.

7. A spring-loaded powder dispersion system as set forth in claim 6, wherein the elastically deformable housing is configured such that a compartment containing a specific powder can be manually removed from the elastically deformable housing and replaced with another compartment containing a different specific powder.

8. A spring-loaded powder dispersion system as set forth in claim 1, wherein the spring-loaded dispersion system is formed such that it is detachably attachable with a sole of a shoe or such that it is permanently attachable with a sole of a shoe.

9. A spring-loaded powder dispersion system as set forth in claim 1, wherein when the spring-loaded powder dispersion system is actuated, the springing means, the elastically deformable housing, the air passage portion, and the top portion are configured to generate an airflow through the spring-loaded powder dispersion system, thereby significantly preventing moisture from penetrating into the elastically deformable housing.

10. A spring-loaded powder dispersion system as set forth in claim 1, wherein a height of the top portion is less than about 1/16 of an inch, a height of the air passage portion is less than about ⅛ of an inch, a height of the elastically deformable housing is less than about ⅛ of an inch, and a height of the springing means is less than about ¼ of an inch.

11. A spring-loaded powder dispersion system as set forth in claim 1, where a moisture absorbent portion is attached above the top portion, whereby the moisture absorbent portion reduces an amount of moisture penetrating into the spring-loaded powder dispersion system.

12. A spring-loaded powder dispersion system as set forth in claim 11, where the moisture absorbent portion has a center aperture, the center aperture allows free passage of air and powder from the spring-loaded powder dispersion system into the foot compartment of a shoe.

13. A spring-loaded powder dispersion system as set forth in claim 12, wherein the moisture absorbent portion is filled with a highly absorbent material surrounded by a hydrophobic material in order to absorb and retain a substantial part of any moisture generated by the foot.

14. A spring-loaded powder dispersion system as set forth in claim 1, wherein the spring-loaded powder dispersion system comprises a pair of spring-loaded powder dispersion systems, the pair having a front system and a rear system, with the front system having a first springing means with a first bottom face and the rear system having a second springing means with a second bottom face.

15. A spring-loaded powder dispersion system as set forth in claim 14, where the first springing means and the elastically deformable housing have a springing diameter and a housing diameter, and where the springing diameter is greater than the housing diameter.

16. A spring-loaded powder dispersion system as set forth in claim 15, where the front system and rear system have a front height and a rear height, and where the rear height is greater than the front height.

17. A spring-loaded powder dispersion system as set forth in claim 16, wherein the first bottom face and the second bottom face have a layer of adhesive formed thereon, and where the first springing means is adhered with a top surface of a sole of a shoe near a front end thereof and a second springing means is adhered with the top surface of the sole of the shoe near a rear end thereof.

18. A spring-loaded powder dispersion system as set forth in claim 1, where the springing means is coated with an anticorrosive material.

19. A spring-loaded powder dispersion system as set forth in claim 2, wherein the specific powder includes a powder selected from a group consisting of an anti-fungal powder, an anti-odor powder, a medicinal powder, and a scented powder.

20. A spring-loaded powder dispersion system as set forth in claim 19, wherein the top portion is substantially circular in shape and the breathable aperture is made of a porous material containing micro-holes.

21. A spring-loaded powder dispersion system as set forth in claim 20, wherein the elastically deformable housing is constructed of plastic and comprises a plurality of compartments.

22. A spring-loaded powder dispersion system as set forth in claim 21, wherein any compartment is interchangeable with any other compartment in the plurality of compartments, and where each compartment contains a powder delivery medium with a specific powder, where the specific powder of a compartment is different from the specific powder of other compartments, and where a compartment with a specific powder content may be placed in the elastically deformable housing at any position.

23. A spring-loaded powder dispersion system as set forth in claim 22, wherein the elastically deformable housing is configured such that a compartment containing a specific powder can be manually removed from the elastically deformable housing and replaced with another compartment containing a different specific powder.

24. A spring-loaded powder dispersion system as set forth in claim 23, wherein the spring-loaded dispersion system is formed such that it is detachably attachable with a sole of a shoe or such that it is permanently attachable with a sole of a shoe.

25. A spring-loaded powder dispersion system as set forth in claim 24, wherein when the spring-loaded powder dispersion system is actuated, the springing means, the elastically deformable housing, the air passage portion, and the top portion are configured to generate an airflow through the spring-loaded powder dispersion system, thereby significantly preventing moisture from penetrating into the elastically deformable housing.

26. A spring-loaded powder dispersion system as set forth in claim 25, wherein a height of the top portion is less than about 1/16 of an inch, a height of the air passage portion is less than about ⅛ of an inch, a height of the elastically deformable housing is less than about ⅛ of an inch, and a height of the springing means is less than about ¼ of an inch.

27. A spring-loaded powder dispersion system as set forth in claim 26, where a moisture absorbent portion is attached above the top portion, whereby the moisture absorbent portion reduces an amount of moisture penetrating into the spring-loaded powder dispersion system.

28. A spring-loaded powder dispersion system as set forth in claim 27, where the moisture absorbent portion has a center aperture, the center aperture allows free passage of air and powder from the spring-loaded powder dispersion system into the foot compartment of a shoe.

29. A spring-loaded powder dispersion system as set forth in claim 28, wherein the moisture absorbent portion is filled with a highly absorbent material surrounded by a hydrophobic material in order to absorb and retain a substantial part of any moisture generated by the foot.

30. A spring-loaded powder dispersion system as set forth in claim 29, wherein the spring-loaded powder dispersion system comprises a pair of spring-loaded powder dispersion systems, the pair having a front system and a rear system, with the front system having a first springing means with a first bottom face and the rear system having a second springing means with a second bottom face.

31. A spring-loaded powder dispersion system as set forth in claim 30, where the first springing means and the elastically deformable housing have a springing diameter and a housing diameter, and where the springing diameter is greater than the housing diameter.

32. A spring-loaded powder dispersion system as set forth in claim 31, where the front system and rear system have a front height and a rear height, and where the rear height is greater than the front height.

33. A spring-loaded powder dispersion system as set forth in claim 32, wherein the first bottom face and the second bottom face have a layer of adhesive formed thereon, and where the first springing means is adhered with a top surface of a sole of a shoe near a front end thereof and a second springing means is adhered with the top surface of the sole of the shoe near a rear end thereof.

34. A spring-loaded powder dispersion system as set forth in claim 33, where the springing means is coated with an anticorrosive material.

35. A powder dispersion system for dispersing powder into a foot compartment of a shoe, where the shoe contains a springing means imbedded into an interior of a sole of the shoe, the powder dispersion system comprising:
a top portion having a breathable aperture therein;
an air passage portion having elastically deformable side walls, an open top part, and an open bottom part, where the open top part and the open bottom part are substantially separated from one another, with the open top part in fluid communication with the breathable aperture of the top portion, and wherein the air passage portion selectively allows for air to be stored in the air passage portion or for air to pass through the air passage portion;
an elastically deformable housing having a top portion and a bottom portion, with the top portion of the elastically deformable housing in air-permeable communication with the open bottom part of the air passage portion, the elastically deformable housing containing a powder delivery medium; and
a springing means embedded into an interior of a sole of a shoe, the springing means being in fluid communication with the bottom portion of the elastically deformable housing, whereby when the powder dispersion system is actuated, the springing means moves the elastically deformable housing, causing the powder delivery medium to release a specific powder which is then passed from the elastically deformable housing and through the breathable aperture into a foot compartment of a shoe where it is distributed about a foot.

36. A powder dispersion system as set forth in claim 35, where the springing means is coated with an anticorrosive material.

37. A spring-loaded powder dispersion system for dispersing powder into a foot compartment of a shoe, the spring-loaded dispersion system comprising:
a top portion having a breathable aperture therein;
an air passage portion having elastically deformable side walls, an open top part, and an open bottom part, where the open top part and the open bottom part are substantially separated from one another, with the open top part in fluid communication with the breathable aperture of the top portion, and wherein the air passage portion selectively allows for air to be stored in the air passage portion or for air to pass through the air passage portion;
an elastically deformable housing having a top portion and a bottom portion, with the top portion of the housing in air-permeable communication with the open bottom part of the air passage portion, the housing containing a power delivery medium and a springing means; whereby when the spring-loaded powder dispersion system is actuated, the springing means pushes air through the elastically deformable housing, causing the powder delivery medium to release a specific powder, which is passed from the elastically deformable housing through the breathable aperture into a foot compartment of a shoe where it is distributed about a foot.

38. A spring-loaded powder dispersion system as set forth in claim 37, where the springing means imbedded into the elastically deformable housing is coated with an anticorrosive material.

39. A method for forming a spring-loaded powder dispersion system, comprising acts of:
selecting a top portion with a breathable aperture;
selecting an air passage portion having elastically deformable side walls, an open top part, and an open bottom part, where the open top part and the open bottom part are substantially separated from one another;
attaching the open top part of the air passage portion with the breathable aperture of the top portion;
selecting an elastically deformable housing having a top portion, a bottom portion, and containing a powder delivery medium;
attaching the top portion of the elastically deformable housing with the open bottom part of the air passage portion;
selecting a springing means; and
attaching the spring means with the bottom portion of the elastically deformable housing, whereby when the spring-loaded powder dispersion system is actuated, the springing means moves the elastically deformable housing, causing the powder delivery medium to release a specific powder which is passed from the elastically deformable housing through the breathable aperture into a foot compartment of a shoe where it is distributed about a foot.

40. A method for forming a spring-loaded powder dispersion system as set forth in claim 39, further comprising acts of selecting a moisture absorbent portion, and attaching the moisture absorbent portion with the top portion, whereby the moisture absorbent portion reduces an amount of moisture penetrating into the spring-loaded powder dispersion system.

41. A method for forming a spring-loaded powder dispersion system as set forth in claim 40, further comprising an act of adding an additional spring-loaded powder dispersion system, thereby comprising a pair of spring-loaded powder dispersion systems, the pair having a front system and a rear system, with the front system having a first springing means with a first bottom face and the rear system having a second springing means with a second bottom face.

42. A method for forming a spring-loaded powder dispersion system as set forth in claim 41, wherein in the act of adding an additional spring-loaded powder dispersion system, the first bottom face and the second bottom face have a layer of adhesive formed thereon, and where the first springing means is attached with a top surface of a sole of a shoe near a front end thereof and a second springing means is attached with the top surface of the sole of the shoe near a rear end thereof.
